(19) United States
(12) Patent Application Publication
(10) Pub. No.: US 2025/0296924 A1
(43) Pub. Date: Sep. 25, 2025

(54) PROCESS FOR PRODUCING POLYHYDROXY ESTERS OF NICOTINIC ACID

(71) Applicant: BioHope (Suzhou) Pharmatech Co., Ltd, Suzhou (CN)

(72) Inventors: Songzhou Hu, Princeton, NJ (US); Zhen Song, Shanghai (CN)

(73) Assignee: BioHope (Suzhou) Pharmatech Co., Ltd, Suzhou (CN)

(21) Appl. No.: 18/614,226

(22) Filed: Mar. 22, 2024

(51) Int. Cl. C07D 401/14

(52) U.S. Cl. CPC C07D 401/14

(57) ABSTRACT

There is disclosed a process for the production of polyhydroxy esters of nicotinic acid by carrying out the esterification reaction of polyhydroxy alcohol and nicotinic acid in the presence of 1-alkylimidazole.

### TECHNICAL FIELD

The invention relates to a process for the production of esters of nicotinic acid and polyhydroxy alcohols. It particularly relates to a process for the production of inositol hexanicotinate in the presence of 1-alkylimidazole.

### BACKGROUND OF THE INVENTION

Polyhydroxy esters of nicotinic acid are important derivatives of niacin, i.e., vitamin B3, an essential ingredient for human health and animal nutrition. Two commercially significant esters are inositol hexanicotinate (I) and sorbitol hexanicotinate (II) of the following formula:

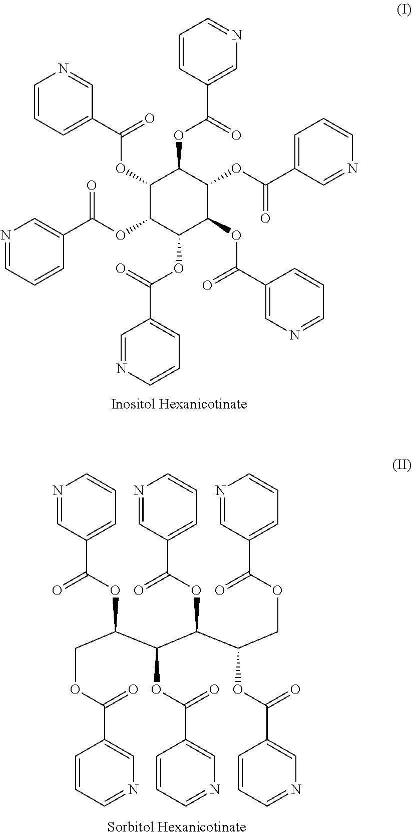 [SMILES: O=C(O[C@H]1[C@H](OC(=O)c2cccnc2)[C@@H](OC(=O)c2cccnc2)[C@H](OC(=O)c2cccnc2)[C@@H](OC(=O)c2cccnc2)[C@H]1OC(=O)c1cccnc1)c1cccnc1 ; O=C(OC[C@H](OC(=O)c1cccnc1)[C@H](OC(=O)c1cccnc1)[C@@H](OC(=O)c1cccnc1)[C@@H](COC(=O)c1cccnc1)OC(=O)c1cccnc1)c1cccnc1]

Inositol hexanicotinate combines the effects of inositol in promoting fat metabolism and lowering blood lipids in the liver and tissues and the effect of niacin in dilating peripheral blood vessels. Inositol hexanicotinate also has broad therapeutical applications as an ideal treatment for hyperlipidemia, Raynaud's disease, and intermittent claudication. It can also be an effective treatment of cardiovascular diseases and various peripheral vascular disorders.

Since inositol hexanicotinate is slowed hydrolyzed into inositol and niacin in the body and has a sustained-release effect, it can be used to overcome adverse effect of administering niacin for extended period of time. In fact, inositol hexanicotinate has been widely used as a no-flush nutritional supplement of niacin.

There are numerous methods for synthesis of esters of nicotinic acid. These methods can be broadly classified into two distinct methods for the synthesis of inositol hexanicotinate.

The first method involves the formation of nicotinyl chloride as an intermediate by means of an activating agent and its subsequent reaction with inositol to form the product of inositol hexanicotinate of formula (I) in two steps as shown in the following reaction scheme:

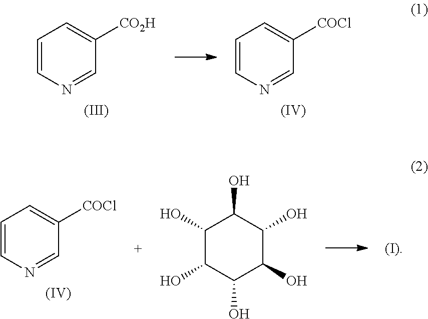

The second method relates to direct esterification of inositol with nicotinic acid by means of an activating agent in one step as shown in the following reaction scheme:

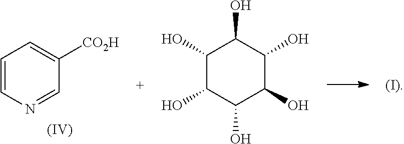

U.S. Pat. No. 3,557,130 discloses a method for producing colorless esters of pyridine carboxylic acids. In this process, pyridine carboxylic acid, particularly nicotinic acid, is suspended in pyridine and reacted with phosphorus oxychloride to form nicotinyl chloride, which is then reacted with inositol to yield inositol hexanicotinate. The reaction mixture is poured into water to precipitate the product. The product is then isolated by filtration and washing with water and acetone or alcohol.

GB 932,079 discloses a process for the preparation of sorbitol hexanicotinate, wherein nicotinyl chloride hydrochloride and sorbitol are reacted in pyridine and the product is isolated by precipitation from water.

EP 0019260 discloses a process for the preparation of D-glucitol hexanicotinate, i.e., sorbitol hexanicotinate, wherein nicotinic acid is reacted with D-glucitol, using nicotinamide as the catalyst, in the presence of phosphorus oxychloride, and in pyridine.

CN 102964298A discloses a process for the production of mannitol hexanicotinate by reacting nicotinic acid in pyridine with thionyl chloride to form nicotinyl chloride, which is then reacted with mannitol to yield mannitol hexanicotinate. After purification by using dilute acid, the product was obtained in a yield of about 66%.

CN 103819399B discloses a method for the production of mannitol hexanicotinate by reacting nicotinic acid in pyridine with phosphorus oxychloride to form nicotinyl chloride and then with mannitol to form mannitol hexanicotinate in a yield of 68.4%. After purification, the overall yield of mannitol hexanicotinate was reduced to 50.2%.

The processes disclosed in the prior art invariably generate an aqueous waste solution containing pyridine and byproducts. CN 102627601 B discloses an improved method for recycling the pyridine in the production of inositol hexanicotinate. In the disclosed method, pyridine is distilled from the aqueous solution by adding sodium hydroxide and repeatedly dried by using solid sodium hydroxide and finally distilled.

These processes are disadvantageous as they use pyridine as scavenging base and solvent. Pyridine is obnoxious and its use as a solvent poses environmental hazards and creates occupational problems.

CN 1546676 discloses an enzymatic process for the production of inositol hexanicotinate, wherein anhydrous tert-butanol is used as a solvent for the reaction of nicotinic acid and inositol in the presence of Novozyme 435. However, the reaction is carried out in a highly diluted solution of inositol (1-2 mmol, 0.18 g-0.36 g/L) and nicotinic acid (6 mmol, 0.74 g/L) and in the presence of a large amount of lipase (5 g/L). It is disadvantageous to use such a highly dilute solution and large amount of enzyme for an industrial process.

CN 113493409A attempts to overcome these disadvantages by disclosing a non-pyridine process for the production of inositol hexanicotinate and sorbitol hexanicotinate. In this process, carbonyl diimidazole is used to react with nicotinic acid in a non-protonic solvent to form nicotinyl imidazole as an intermediate which then condenses with inositol to form the product. Although the process can be carried out in the absence of pyridine, the use of carbonyl diimidazole presents its own industrial challenges. First, carbonyl diimidazole is available at great cost. Second, the byproduct of imidazole is difficult to recover. Third, the process requires the use of large amounts of organic solvents. As a result, the process is economically disadvantageous.

It is an object of the present invention to disclose a process for the production of polyhydroxy esters of nicotinic acid that ameliorates the disadvantages of the known processes.

It is another object of the present invention to eliminate the use of pyridine as a reaction solvent in the production of polyhydroxy ester of nicotinic acid.

### SUMMARY OF THE INVENTION

The invention discloses a process for the production of polyhydroxy esters of nicotinic acid by reacting polyhydroxy alcohol and nicotinic acid in the presence of 1-alkylimidazole. It particularly relates to a process for the production of polyhydroxy esters of nicotinic acid by reacting polyhydroxy alcohol and nicotinic acid in an ionic liquid based on 1-alkylimidazole.

### DETAILED DESCRIPTION OF THE INVENTION

The present invention relates to a process for the production of polyhydroxy esters of nicotinic acid. In particular, it relates to a process for the production of polyhydroxy esters of nicotinic acid without using pyridine as a reaction solvent.

The present invention is accomplished by carrying out the esterification reaction of a polyhydroxy alcohol with nicotinic acid or its derivatives by means of an activating agent in the presence of 1-alkylimidazole of the following formula:

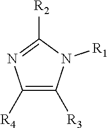

wherein R₁ can be linear, branched, cyclic, or substituted C₁-C₁₂ alkyl, or aryl, and wherein R₂, R₃, and R₄ can each be, independently of one another, hydrogen or linear, branched, cyclic, or substituted C₁-C₁₂ alkyl or aryl.

Exemplary “alkyl” groups include methyl, ethyl, propyl, isopropyl, cyclopropyl, n-butyl, t-butyl, isobutyl, cyclobutyl, cyclopropylmethyl, pentyl, isopentyl, hexyl, isohexyl, heptyl, 4,4-dimethylpentyl, octyl, 2,2,4-trimethylpentyl, nonyl, decyl, undecyl, dodecyl, 2-methoxyethyl, 2-ethoxyethyl, 3-methoxypropyl, 3-ethoxypropyl, 2-dimethylaminoethyl, 3-dimethylaminopropyl, 2-chloroethyl, 3-chloropropyl, and the like.

Exemplary “aryl” groups include phenyl, substituted phenyl, naphthyl, and substituted napththyl, pyridyl, substituted pyridyl, and the like.

Preferably, the present invention is accomplished by carrying out the esterification reaction of a polyhydroxy alcohol with nicotinic acid or its derivatives by means of an activating agent in an ionic liquid based on 1-alkylimidazole of the formula:

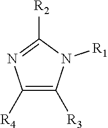

wherein R₁ can be linear, branched, cyclic, or substituted C₁-C₁₂ alkyl, or aryl, and wherein R₂, R₃, and R₄ can each be, independently of one another, hydrogen or linear, branched, cyclic. or substituted C₁-C₁₂ alkyl, or aryl.

More preferably, the 1-alkylimidazole is selected from the group consisting of 1-methylimidazole, 1-ethylimidazole, 1-propylimidazole, 1-butylimidazole, 1-isobutylimidazole, and a mixture thereof.

The polyhydroxy alcohols are selected from the group consisting of inositol, sorbitol (also known as D-glucitol), mannitol, glycerol, xylitol, pentaerythritol, and a mixture thereof. Preferably, the polyhydroxy alcohol is inositol or myo-inositol, wherein the product is inositol hexanicotinate.

The polyhydroxy ester of nicotinic acid is formed by means of an activating agent. Suitable activating agents are selected from the group consisting of phosphorus oxychloride, phosphorus trichloride, phosphorus pentachloride, thionyl chloride, sulfuryl dichloride, phosgene, diphosgene, triphosgene, alkyl sulfonyl chloride, aryl sulfonyl chloride, and a mixture thereof. The alkyl sulfonyl chloride can be Ci-C₁₂ linear, cyclic, or branched alkyl sulfonyl chloride. The aryl sulfonyl chloride can be benzenesulfonyl chloride or substituted phenyl sulfonyl chloride, naphthyl sulfonyl chloride or substituted naphthyl sulfonyl chloride, pyridyl sulfonyl chloride, and a mixture thereof.

It is surprising and unexpected to find that nicotinic acid can form a salt with 1-alkylimidazole of low melting point that is characteristic of an ionic liquid. It is even more surprising and unexpected to find that the ionic liquid is miscible with the 1-alkylimidazole and can form a homogenous solution. This ionic liquid solution can be advantageously used to produce the polyhydroxy ester of nicotinic acid in the esterification reaction of nicotinic acid with a polyhydroxy alcohol.

In the process of the present invention, the esterification reaction can be carried out by mixing the reactants in 1-alkylimidazole in any order. Preferably, the reaction can be carried out by mixing nicotinic acid with 1-alkylimidazole to form an ionic liquid solution, followed by adding an activating agent to form either nicotinyl chloride or a mixed anhydride of nicotinic and a sulfonic acid as an intermediate. After the formation of the intermediate, a polyhydroxy alcohol is then added to form the polyhydroxy ester of nicotinic acid.

More preferably, the esterification reaction can be carried out by mixing nicotinic acid and a polyhydroxy alcohol in 1-alkylimidazole, followed by the addition of an activating agent to form the polyhydroxy ester of nicotinic acid.

In addition, nicotinyl chloride hydrochloride, an activated derivative of nicotinic acid, may be reacted with a polyhydroxy alcohol in 1-alkylimidazole to produce the polyhydroxy ester of nicotinic acid. In this case, addition of an activating agent is not necessary, but can be applied to ensure the completion of the reaction.

The amount of 1-alkylimidazole used in the reaction is not limited, relative to the amount of either polyhydroxy alcohol or nicotinic acid. Preferably, 1-alkylimidazole is used to form an ionic liquid with nicotinic acid and as a reaction solvent. In addition, 1-alkylimidazole can also be used as an acid scavenging agent to neutralize the acid liberated by the activating agent during the esterification reaction. The acids liberated from the activating agents can be hydrochloric acid or alkyl sulfonic acid or aryl sulfonic acid.

The process of present invention provides an added advantage in that 1-alkylimidazole can form an ionic liquid with the acid, for example, hydrochloric acid, methane sulfonic acid, benzene sulfonic acid, or p-toluene sulfonic acid, liberated from the activating agent during the esterification reaction. The formation of an ionic liquid in the process of the present invention, rather than a crystalline solid of pyridine salt, increases the fluidity of the reaction mixture, enhances the heat transfer, and ameliorates the formation of hot spots.

In some instances, it can be useful to use a non-protonic solvent to further increase the fluidity of the reactants and product in the reaction mixture. Suitable non-protonic solvents can be selected from the group consisting of ketones of C₃-C₁₂, nitriles of C₂-C₁₂, esters, ethers, amides, dialkyl carbonates, sulfoxides, sulfones, halogenated alkanes, aliphatics, halogenated aromatics, and aromatics; wherein the alkyl is C₁-C₁₂ and wherein the halogen is fluoro, chloro, bromo, or a mixture thereof, and wherein the aromatics are selected from the group consisting of phenyl, naphthalene, and their substituted derivatives. Preferably, the non-protonic solvent is toluene or xylenes.

The temperature for the esterification reaction can be from 10° C. to 150° C. Preferably, the reaction is performed at a temperature from 20° C. to 100° C. More preferably, the reaction is performed at a temperature from 30° C. to 90° C. Most preferably, the reaction is performed at a temperature from 40° C. to 80° C.

After the esterification reaction is completed, the polyhydroxy ester of nicotinic acid can be isolated by any suitable method. Preferably, the reaction mixture is diluted with water to precipitate the ester, which can be isolated by a solid-liquid separation. After washing with water, a colorless product can be obtained.

The yield of the polyhydroxy ester of nicotinic acid can be at least 85%, preferably, at least 90%, more preferably at least 95%, on the molar basis of the polyhydroxy alcohol.

To further reduce the residual amount of 1-alkylimidazole from the isolated product, it is found to be effective to use a dilute solution of an acid to wash the solid product. The acid can be selected from the group consisting of an inorganic acid, an organic carboxylic acid, and a mixture thereof. Suitable inorganic acids are hydrochloric acid, sulfuric acid, nitric acid, phosphoric acid, phosphorous acid, and a mixture thereof. Suitable organic carboxylic acids are formic acid, acetic acid, propionic acid, butyric acid, glycolic acid, oxalic acid, malic acid, tartaric acid, citric acid, fumaric acid, maleic acid, succinic acid, malonic acid, and a mixture thereof.

After the separation of polyhydroxy ester of nicotinic acid, the mother liquor solution contains salts of 1-alkylimidazole. The 1-alkylimidazole in the mother liquor solution can be recovered by any method known to one skilled in the art, such as distillation and extraction, and reused for the production of the polyhydroxy ester of nicotinic acid.

When alkyl sulfonyl chloride or aryl sulfonyl chloride is used in the esterification reaction, the 1-alkylimidazole salt of the corresponding alkyl sulfonic acid or aryl sulfonic acid can be converted to their alkali salt by reacting with an alkali hydroxide, wherein the alkali is lithium, sodium, potassium, or a mixture thereof. The alkali sulfonate can be readily recovered from the aqueous solution by concentration and crystallization, and then converted to its corresponding sulfonyl chloride by any method known to one skilled in the art.

It is understood that the polyhydroxy ester of nicotinic acid, produced by the process of the present invention, contains no detectable amount of pyridine, since pyridine is not used in the entire process. On the other hand, the product of the polyhydroxy ester, produced by the process of the present invention, can contain a trace amount of 1-alkylimidazole.

There are distinct advantages in using 1-alkylimidazole in the production of polyhydroxy ester of nicotinic acid over the use of pyridine. First, nicotinic acid forms a clear solution in 1-alkylimidazole, but a thick suspension in pyridine. A solution is much easier to handle than a solid suspension. Second, the byproduct of 1-alkylimidazole hydrochloride can be maintained as an ionic liquid by choosing appropriate reaction temperature to increase the fluidity of the reaction mixture. In contrast, pyridine hydrochloride has much higher melting point at 145-147° C. than the boiling point of 115° C. As a result, pyridine hydrochloride is formed as a crystalline solid in the reaction mixture. Third, the polyhydroxy ester of nicotinic acid, in particular, inositol hexanicotinate, can be obtained as a colorless product, thus greatly simplifying the final purification of the product. Fourth, the process of the present invention ameliorates obnoxious odor of pyridine inherently present in the pyridine process.

The process according to the present invention can be carried out discontinuously, semi-continuously, or continuously.

### EXAMPLES

The following examples will illustrate the practice of this invention but are not intended to limit its scope.

### Example 1

To a 50 mL round bottom flask were added 12.3 g of nicotinic acid and 8.2 g of 1-methylimidazole. The mixture was heated to 82° C. to obtain a clear solution. Upon cooling, crystalline salt started to appear at 75° C. Further cooling resulted in the formation of crystalline solid. To the solid was added 4.5 g of 1-methylimidazole. Upon warming to about 40° C., a clear solution was formed.

### Example 2

To a 500 mL round bottom flask were added 80 g of 1-methylimidazole, 9 g of inositol, and 42 g of nicotinic acid. The mixture was stirred and warmed to 35° C. To the stirred mixture was added dropwise 45 mL of benzenesulfonyl chloride, while the temperature was allowed to rise to 80-85° C. After the suspension was stirred at this temperature for additional 30 minutes, 200 mL of deionized water was added to the flask to yield crystalline solid product. The suspension was stirred and cooled to 20° C. The suspension was then filtered and washed extensively with deionized water to yield a colorless crystalline product. After drying, 40.0 g of inositol hexanicotinate was obtained in a molar yield of 98.8% on the basis of inositol.

### Example 3

To a 250 mL round bottom flask were added 25 g of 1-butylimidazole, 1.8 g of inositol, and 9 g of nicotinic acid. To the stirred mixture at 14° C. was added dropwise 9 mL of benzenesulfonyl chloride, while the temperature was allowed to rise to 80-85° C. After the suspension was stirred at this temperature for additional 30 minutes, 75 mL of deionized water was added to the flask to yield crystalline solid product. The suspension was stirred and cooled to 20° C. The suspension was then filtered and washed extensively with deionized water to yield a colorless crystalline product. After drying, 7.8 g of inositol hexanicotinate was obtained in a molar yield of 96.2% on the basis of inositol.

### Example 4

To a 250 mL round bottom flask were added 23 g of 1-methylimidazole, 1.82 g of sorbitol, and 9 g of nicotinic acid. To the stirred mixture at 11° C. was added dropwise 5.4 mL of methanesulfonyl chloride, while the temperature was allowed to rise to 80-85° C. After the suspension was stirred at this temperature for additional 30 minutes, 75 mL of deionized water was added to the flask to yield crystalline solid product. The suspension was stirred and cooled to 20° C. The suspension was then filtered and washed extensively with deionized water to yield a colorless product. After drying, 7.9 g of sorbitol hexanicotinate was obtained in a molar yield of 97.2% on the basis of sorbitol.

### Example 5

To a 500 mL round bottom flask were added 115 g of 1-propylimidazole, 9 g of inositol, and 42 g of nicotinic acid. The mixture was stirred and warmed to 30° C. To the stirred mixture was added dropwise 45 mL of benzenesulfonyl chloride, while the temperature was allowed to rise to 80-85° C. After the suspension was stirred at this temperature for additional 30 minutes, 200 mL of deionized water was added to the flask to yield crystalline solid product. The suspension was stirred and cooled to 20° C. The suspension was then filtered and washed extensively with deionized water to yield a colorless crystalline product. After drying, 40.0 g of inositol hexanicotinate was obtained in a molar yield of 98.8% on the basis of inositol.

### Example 6

To a 250 mL round bottom flask were added 50 g of 1-ethylimidazole, 3.6 g of inositol, and 18 g of nicotinic acid. To the stirred mixture at 14° C. was added 13 g of triphosgene, while the temperature was allowed to rise to 80-85° C. After the suspension was stirred at this temperature for additional 30 minutes, 150 mL of deionized water was added to the flask to yield solid product. The suspension was stirred and cooled to 20° C. The suspension was then filtered and washed extensively with deionized water to yield a crystalline product. After drying, 12.5 g of inositol hexanicotinate was obtained in a molar yield of 77.2% on the basis of inositol.

### Example 7

To a 250 mL round bottom flask were added 42 g of 1-ethylimidazole, 3.6 g of inositol, and 18.2 g of nicotinic acid. To the stirred mixture at 24° C. was added 6.8 mL of phosphorus oxychloride, while the temperature was allowed to rise to 80-85° C. After the suspension was stirred at this temperature for additional 30 minutes, 150 mL of deionized water was added to the flask to yield solid product. The suspension was stirred and cooled to room temperature. The suspension was then filtered and washed extensively with deionized water to yield a crystalline product. After drying, 12.3 g of inositol hexanicotinate was obtained in a molar yield of 75.9% on the basis of inositol.

### Example 8

To a 250 mL round bottom flask were added 42 g of 1-ethylimidazole and 18.2 g of nicotinic acid. To the stirred mixture at 20° C. was added 10.2 mL of thionyl chloride, while the temperature was allowed to rise to 60-70° C. After the solution was stirred at this temperature for additional 2 hours, 3.6 g of inositol was added in a small amount, while the temperature was kept between 60-70° C. After the addition, the temperature was increased to 80° C. and maintained for 2 hours. Then, 150 mL of deionized water was added to the flask to yield solid product. After the suspension was stirred and cooled to room temperature, the suspension was filtered and washed extensively with deionized water to yield a crystalline product. After drying, 12.6 g of inositol hexanicotinate was obtained in a molar yield of 77.8% on the basis of inositol.

### Example 9

To a 250 mL round bottom flask were added 42 g of 1-ethylimidazole, 3.6 g of inositol, and 18.2 g of nicotinic acid. To the stirred mixture at 20° C. was added 26.7 g of p-toluenesulfonyl chloride, while the temperature was allowed to rise to 80-85° C. After the suspension was stirred at this temperature for additional 30 minutes, 150 mL of deionized water was added to the flask to yield crystalline solid product. The suspension was stirred and cooled to room temperature. The suspension was then filtered and washed extensively with deionized water to yield a colorless crystalline product. After drying, 15.9 g of inositol hexanicotinate was obtained in a molar yield of 98.1% on the basis of inositol.

### Example 10

To a 250 mL round bottom flask were added 23 g of 1-methylimidazole, 1.82 g of mannitol, and 9 g of nicotinic acid. To the stirred mixture at 23° C. was added dropwise 5.4 mL of methanesulfonyl chloride, while the temperature was allowed to rise to 80-85° C. After the suspension was stirred at this temperature for additional 30 minutes, 75 mL of deionized water was added to the flask to yield crystalline solid product. The suspension was stirred and cooled to 20° C. The suspension was then filtered and washed extensively with deionized water to yield a colorless product. After drying, 7.8 g of mannitol hexanicotinate was obtained in a molar yield of 96.1% on the basis of mannitol.

### Example 11

To a 250 mL round bottom flask were added 21 g of 1-methylimidazole, 15 g of toluene, 1.8 g of inositol, and 9.2 g of nicotinic acid. To the stirred mixture at 8° C. was added dropwise 8.9 mL of benzenesulfonyl chloride, while the temperature was allowed to rise to 80-85° C. After the suspension was stirred at this temperature for additional 30 minutes, 75 mL of deionized water was added to the flask to yield crystalline solid product. The suspension was stirred and cooled to 20° C. The suspension was then filtered and washed extensively with deionized water to yield a colorless product. After drying, 7.9 g of inositol hexanicotinate was obtained in a molar yield of 97.5% on the basis of inositol.

It will be understood that the foregoing examples and explanation are for illustrative purposes only and that various modifications of the present invention will be self-evident to those skilled in the art. Such modifications are to be included within the spirit and purview of this application and the scope of the appended claims.

What is claimed is:

1. A process for producing a polyhydroxy ester of nicotinic acid comprises reacting a polyhydroxy alcohol and nicotinic acid by means of an activating agent in the presence of 1-alkylimidazole of the following structure: 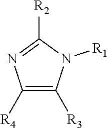
wherein R₁ can be linear, branched, or substituted C₁-C₁₂ alkyl, or aryl, and wherein R₂, R₃, and R₄ can each be, independently of one another, hydrogen or linear, branched, or substituted C₁-C₁₂ alkyl, or aryl.

2. The process according to claim 1, wherein the polyhydroxy alcohol is selected from the group consisting of inositol, myo-inositol, sorbitol, mannitol, glycerol, erythritol, and a mixture thereof.

3. The process according to claim 1, wherein the activating agent is selected from the group consisting of phosphorus oxychloride, phosphorus trichloride, phosphorus pentachloride, thionyl chloride, sulfuryl dichloride, phosgene, diphosgene, triphosgene, alkyl sulfonyl chloride, aryl sulfonyl chloride, and a mixture thereof.

4. The process according to claim 1, wherein the polyhydroxy ester of nicotinic acid is inositol hexanicotinate.

5. The process according to claim 1, wherein the polyhydroxy ester of nicotinic acid is sorbitol hexanicotinate.

6. The process according to claim 1, wherein the polyhydroxy ester of nicotinic acid is mannitol hexanicotinate

7. The process according to claim 1, wherein the polyhydroxy ester of nicotinic acid contains no detectable amount of pyridine.

8. The process according to claim 1, wherein the polyhydroxy ester of nicotinic acid contains a trace amount of 1-alkylimidazole.

9. The process according to claim 1, wherein the 1-alkylimidazole is 1-methylimidazole

10. The process according to claim 1, wherein the 1-alkylimidazole is 1-ethylimidazole.

11. The process according to claim 1, wherein the 1-alkylimidazole is 1-propylimidazole.

12. The process according to claim 1, wherein the 1-alkylimidazole is 1-butylimidazole.

13. The process according to claim 1, wherein the activating agent is phosphorus oxychloride.

14. The process according to claim 1, wherein the activating agent is methanesulfonyl chloride.

15. The process according to claim 1, wherein the activating agent is benzenesulfonyl chloride.

16. The process according to claim 1, wherein the activating agent is toluenesulfonyl chloride.

17. The process according to claim 1, wherein the activating agent is triphosgene.

18. The process according to claim 1, wherein the activating agent is thionyl chloride.
